(19) United States
(12) Patent Application Publication
(10) Pub. No.: US 2019/0125974 A1
(43) Pub. Date: May 2, 2019

(54) INJECTION DEVICE WITH DOSING RESERVOIR FOR A FRACTION OF SOLUTION

(71) Applicant: OROFINO PHARMACEUTICALS GROUP SRL, Rome (IT)

(72) Inventor: Ernesto OROFINO, Rome (IT)

(21) Appl. No.: 16/093,359

(22) PCT Filed: Apr. 11, 2017
(86) PCT No.: PCT/IB2017/052073

§ 371 Date: Oct. 12, 2018

(30) Foreign Application Priority Data
Apr. 13, 2016 (IT) 102016000037972

(51) Int. Cl. A61M 5/31; A61M 5/315

(52) U.S. Cl. CPC A61M 5/3134; A61M 2005/3143; A61M 2005/3123; A61M 5/31596

(57) ABSTRACT

An injection device (1) for an injectable solution (S) includes one tubular body (3) inside which at least one containment chamber (8,9) for the injectable solution is defined; one dosing reservoir (20) for one fraction of injectable solution (S); at least one plug (6,7) sliding into the tubular body (3) and including a protruding appendix (16) suitable for entering in the dosing reservoir (20) to eject said fraction of solution; one vent outlet (23) to eject said second fraction of solution. The device (100) includes an accessory (260) with one storing cannula (261) engageable with the vent opening (23), and provided with a first end (266) and a second end (267) between which a storing reservoir (261′) for the second fraction of solution is defined. The device (100), that is particularly suitable for injecting very precise and reduced doses of an injectable solution, is for example of the single-chamber or double-chamber pre-filled type.

This invention relates to a device for injecting very precise and reduced doses of an injectable solution.

Hence the invention relates to the technical field of syringes for injections or of cartridges, of the pre-filled type too, with multiple chambers too for injecting a solution which is reconstituted just before being administered.

Patent application number IT2014RM00408, always by this proprietor, represents an example of known syringes of the pre-filled double-chamber type. Such a syringe allows to inject very reduced doses of a reconstituted solution. Such a known syringe includes one vent outlet for ejecting a fraction of solution, such ejection being necessary to enable to reach an end-administration configuration. Such fraction of solution is ejected alongside the injection of the fraction of administration. During the injection step, the physician is thus forced to close or tampon the vent outlet by means of absorbing material to collect the fraction of solution ejected from the syringe. Such operation has some drawbacks: it makes the injection less simple and it forces the physician to come into contact with the pharmaceutical solution.

The need perceived by the sector of the injection devices provided with a vent outlet is to have available devices able to prevent the dispersion of the ejected fraction during the injection of the administration fraction.

The object of this invention is to overcome the problems of the prior art taking into account the needs of the field.

Such an object is obtained by an injection device according to this invention, provided with a storing cannula for containing the ejected fraction.

The solution according to this invention is particularly advantageous as the storing cannula for containing the ejected fraction can be easily applied to the injection device with no need of substantial changes.

The solution according to this invention is generally suitable for injection devices with two front outlet ducts, and it is particularly suitable for injection devices for very precise and reduced doses of an injectable solution (namely syringes for micro-doses for example doses of one millilitre or of one tenth of millilitre).

Such an object is obtained by an injection device according to claim 1. Dependent claims disclose preferred or advantageous embodiments of the device.

The features and the advantages of the injection device according to this invention are made clear from the hereinafter related exemplary but non-limiting disclosure according to the enclosed figures, wherein:

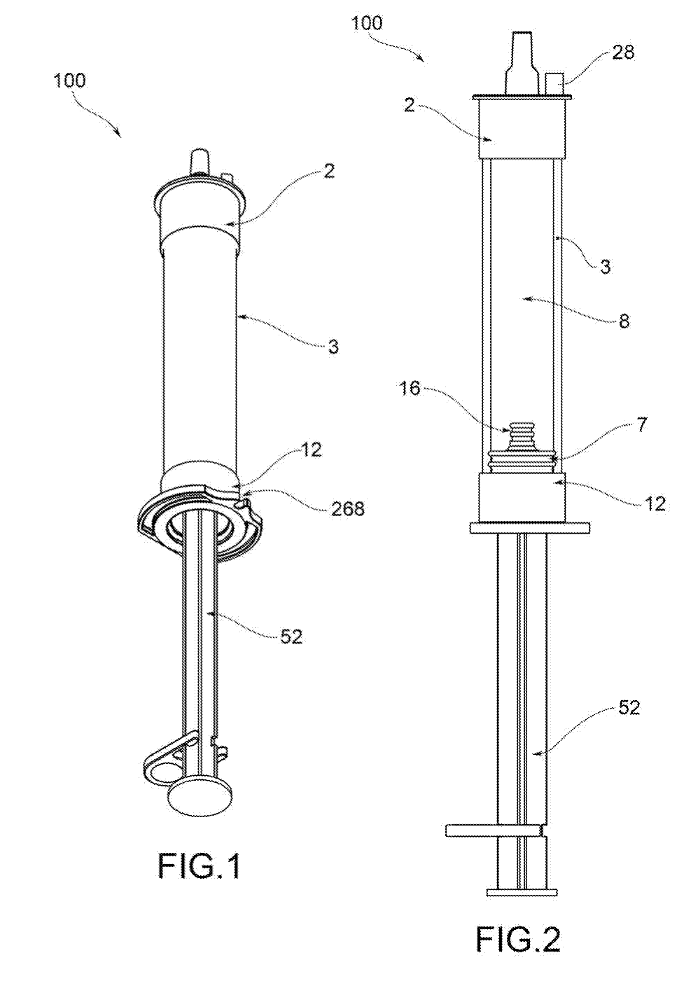

FIGS. 1 and 2 show respectively an axonometric projection view and a cross section view of an injection device according to this invention, in one embodiment;

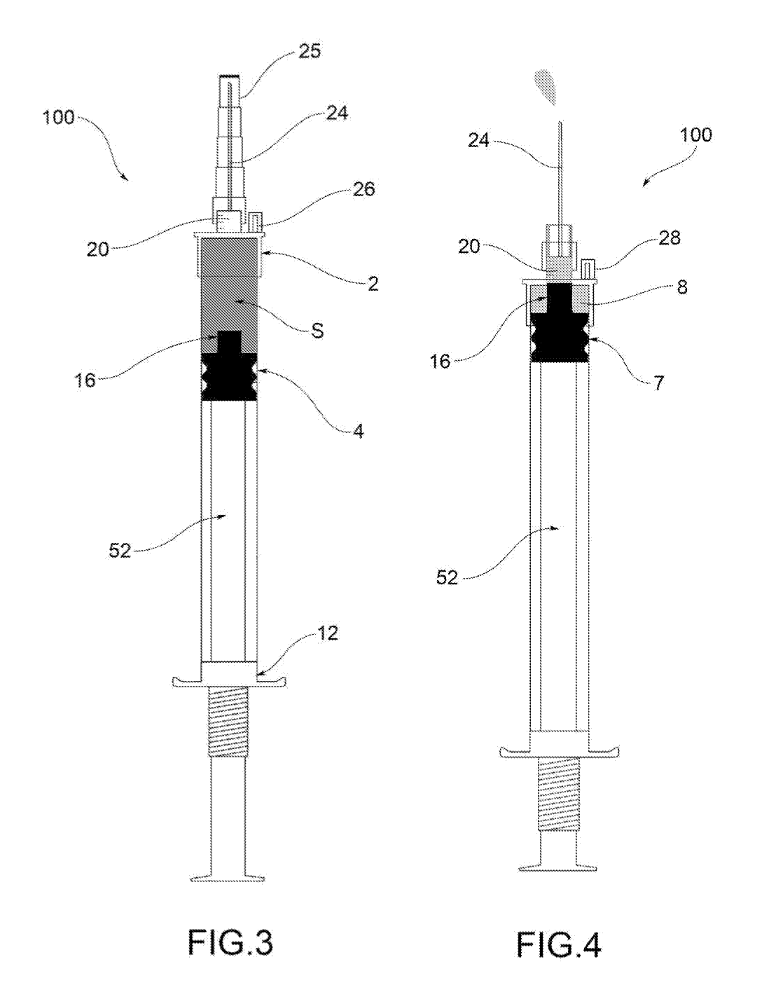

FIGS. 3 and 4 show a cross section view of the device of FIG. 1 respectively in an initial configuration and in a pre-administering configuration;

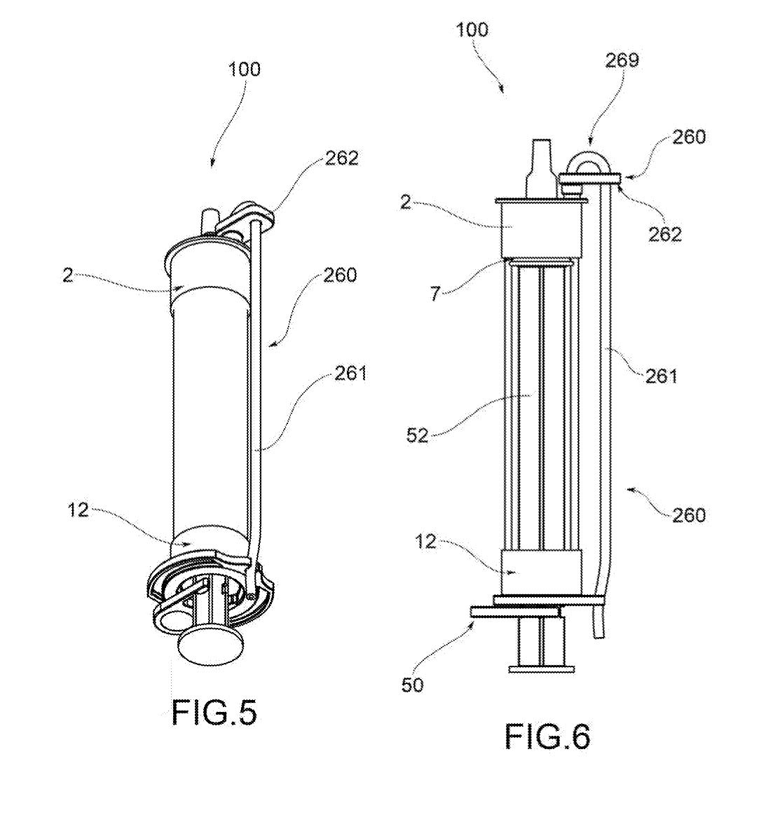

FIGS. 5 and 6 show respectively an axonometric projection view and a cross section view of an injection device provided with one accessory according to this invention;

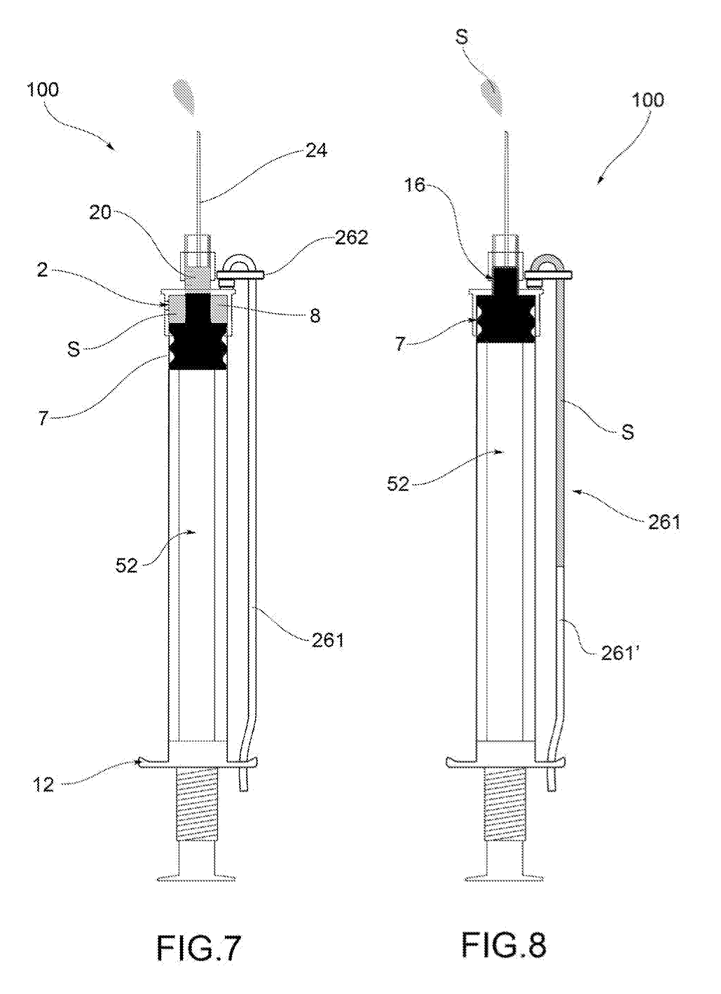

FIGS. 7 and 8 show a cross section view of the device of FIG. 5 respectively in a pre-administering configuration and in an end-administering configuration;

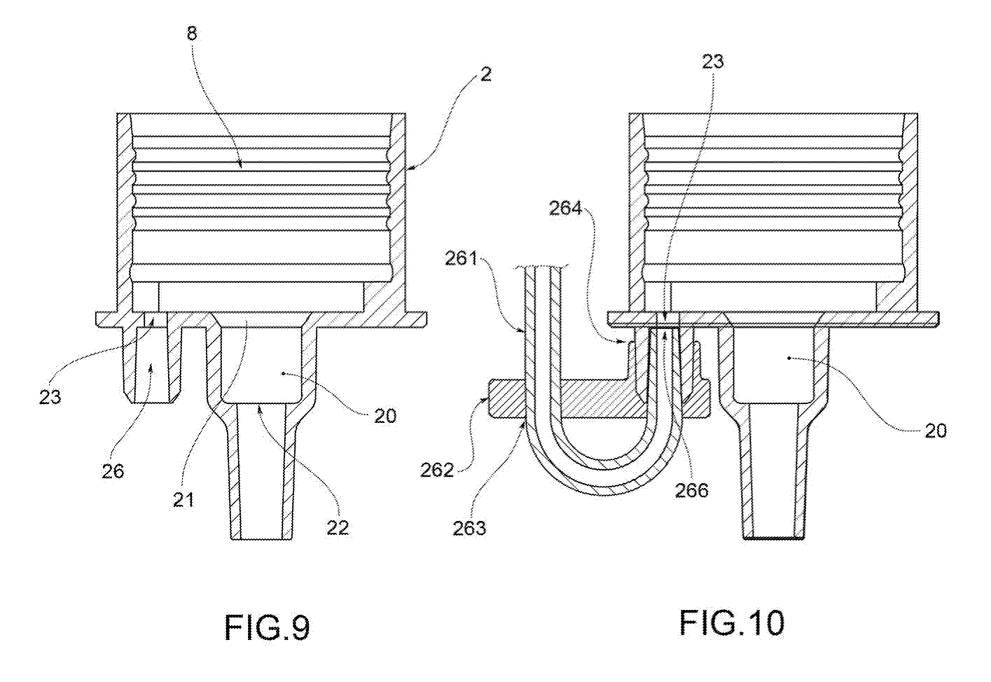

FIGS. 9 and 10 show a cross section view of one detail of the device of FIG. 1 and of the device of FIG. 4 respectively;

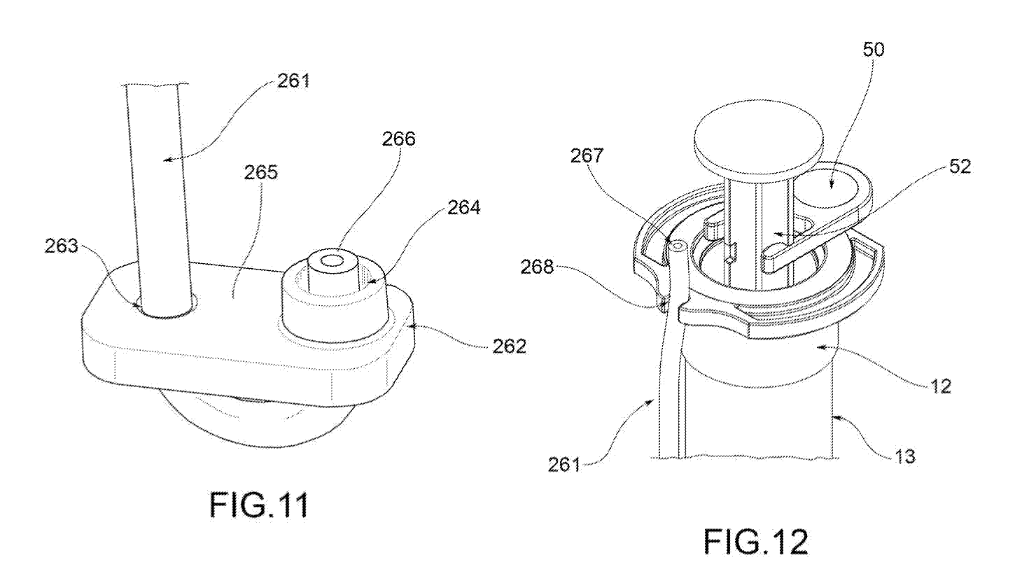

FIG. 11 shows one detail of an accessory according to this invention;

FIG. 12 shows one detail of the device of FIG. 5;

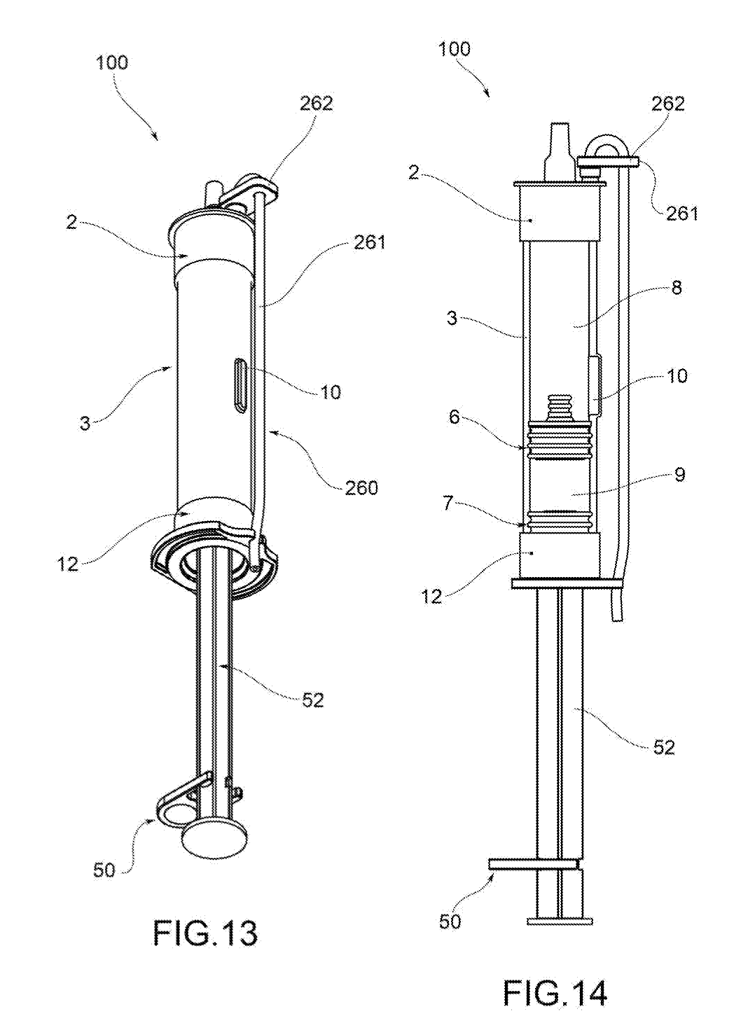

FIGS. 13 and 14 show respectively an axonometric projection view and a cross section view of an injection device according to this invention, in one further embodiment;

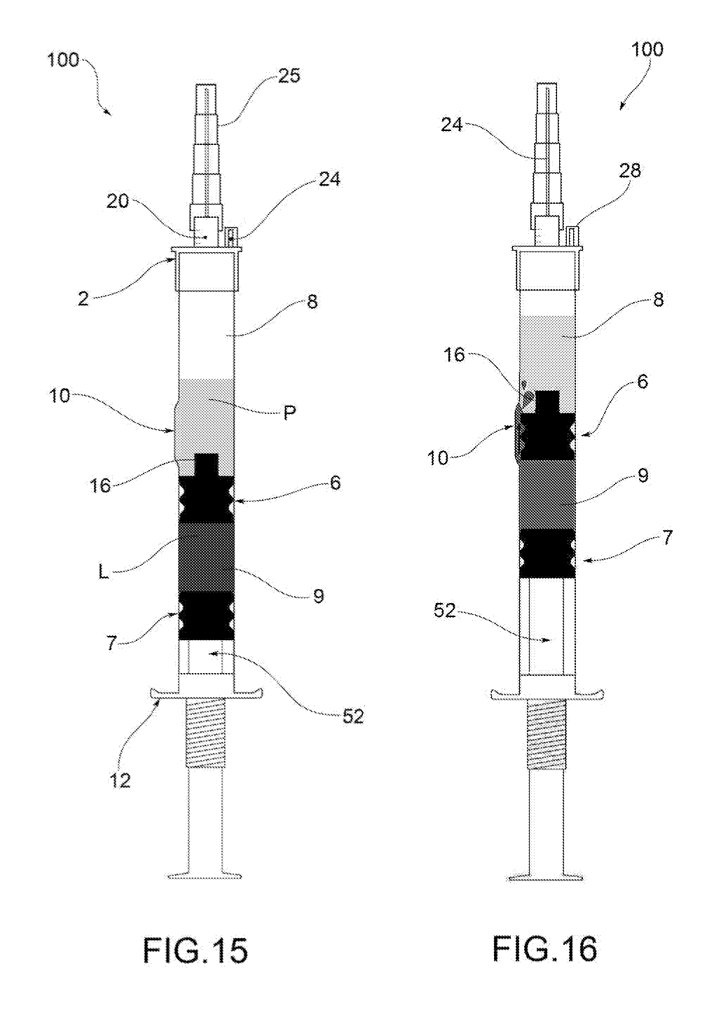

FIGS. 15 and 16 show a cross section view of the device of FIG. 13 respectively in an initial configuration and in a reconstitution configuration of the injectable solution.

In the enclosed figures same or similar elements will be indicated by same reference numbers.

Referring to the enclosed Figures, and in particular to FIGS. 1 and 13 an injection device is indicated by reference number 100.

In FIGS. 1 to 4 one embodiment of an injection device 100 is represented, which in the specific represented example, is embodied by a single-chamber syringe. Preferably, the injection device 100 is of the pre-filled type with an injectable solution S. In one alternative embodiment, the injection device 100 is a pre-filled cartridge.

In FIGS. 13 to 16 a further embodiment of an injection device 100 is represented which in the specific represented example is embodied in a double-chamber pre-filled cartridge. In one alternative embodiment, the injection device is a pre-filled cartridge. The double-chamber injection device is pre-filled with two substances, contained in two originally separated containment chambers, intended to be mixed with each other immediately before being administered to the patient. In order to mix the two substances, the two containment chambers are put in communication with each other in such a way as to enable the reconstitution of the injectable solution. In further embodiments more than two chambers may be provided in the pre-filled injection device.

Preferably, the injection device 100 according to this invention (both in the single-chamber variant and in the double-chamber variant) is suitable for the administration of a very reduced dose of an injectable solution, for example for injecting a quantity of solution smaller than 1 millilitre and preferably of, or almost of, 0.1 millilitre. Preferably the aforesaid dose is smaller than about 1/25 of the injectable solution contained inside the injection device, and preferably of 1/50. For instance, such doses are required to administer antibiotics locally, for example for injecting a cephalosporin antibiotic, such as cefuroxime, into the ocular room of a patient.

Referring to FIG. 1, the injection device 100 includes a containment tubular body 3 closed at the front by a closing element 2 and, at the opposite side or at the back, by a grasping portion 12.

The closing element 2 and the grasping portion 12 are fixed to the tubular body 3 or are integrally made with the tubular body 3.

The tubular body 3 is the containment body of a syringe or of a cartridge, it is suitable for containing injectable substances, and it is preferably made of glass or of a transparent or substantially transparent plastic material. Preferably, the tubular body 3 is made in a single piece.

The injection device 100 includes at least one containment chamber 8 suitable for containing an injectable solution S. In the embodiment variant wherein the injection device 100 is of the pre-filled type, the containment chamber 8 contains an injectable solution S. The injection device 100 includes at least one plug 7 arranged within the tubular body 3, so as to delimit the containment chamber 8.

The plug 7 is for example made of plastic material and is such as to sealingly engage with the inner wall of the tubular body 3.

Plug 7 is suitable to slide inside the tubular body 3 under the action of an external pushing or pulling force. Plug 7 is fixed to a plunger 52 so as to slide inside the tubular body 3 under the action of an external pushing or pulling force.

As shown in FIGS. 3 and 9, the injection device 100 includes a dosing reservoir 20, having one inlet opening 21 and one outlet opening 22. The inlet opening 21 is in communication with the containment chamber 8. In the specific represented example the dosing reservoir 20 is defined within the closing device 2, however in a embodiment variant it may be part of the tubular body 3 itself and it represents a narrowing of the first containment chamber 8.

Preferably, the dosing reservoir 20 is made of transparent or translucent material so that its content can be seen from outside, and even more preferably, it includes a graduated scale visible from outside too.

Preferably, the injection device 100 includes a needle 24 whose internal channel is in communication with fluid with the outlet opening 22 of the dosing reservoir 20.

Preferably, the injection device 100 includes a protective screen 25, or cap, for the needle 24.

Starting from the initial configuration of FIG. 3, under the pushing action of plunger 52, after removing the protective cap 25, it is possible to make the plug 7 slide to reduce the volume of the containment chamber and eject a fraction (henceforth called “first fraction”) of the injectable solution from the tubular body 3 through the outlet opening 22 of the dosing reservoir 20 until it reaches a pre-administering configuration represented in FIGS. 4 and 7.

In the pre-administering configuration:
the plug 7 obstructs the inlet opening of the dosing reservoir 20;
one fraction (henceforth called “second fraction”) of the solution is contained inside the containment chamber 8; and
the dosing reservoir 20 is filled with a fraction (henceforth called “third fraction”) of injectable solution which represents of dose of solution to be administered to the patient.

Preferably the aforesaid third fraction is less than about 1/25 of the injectable solution and preferably equal to about 1/50 of the injectable solution.

Preferably, the aforesaid dosing reservoir 20 has a volume of one millilitre or one tenth of a millilitre. The plug 7 includes a protruding appendix 16 suitable to pass through the inlet opening 21 of the dosing reservoir 20 and enter inside the latter in order to eject the third injectable solution from the dosing reservoir 20. This occurs for and during the administration of the third fraction of solution.

Preferably, the protruding appendix 16 has a cross section equal to the inlet opening of the dosing reservoir 20 or slightly smaller so that it can enter inside the dosing reservoir 20 sealingly engaging with the internal walls of the dosing reservoir 20 while sliding inside the latter.

As shown in FIGS. 3 and 9, the injection device 100 includes a vent opening 23, or vent way, suitable to be passed through by the second fraction of solution for ejecting the second fraction and to allow the protruding appendix 16 to pass through the inlet opening 21 of the dosing reservoir 20. Without the vent outlet 23, the second fraction of solution would make opposition to the plug 7 advancement to pass from the operative configuration of FIG. 4 or 7 to the operative configuration of FIG. 8, called “end-administration configuration”.

Preferably, the vent opening 23 is provided with a vent channel 26.

Preferably, the vent opening 23 and the vent channel 26 are provided in the closing element 2. Preferably, the vent channel 26 is provided in parallel with the dosing reservoir 20.

Preferably, the vent opening 23 is initially closed, for instance by a removable plug 28.

The vent opening 23 is configured in such a way as to be opened starting from configuration of FIG. 4, that is say from the pre-administering configuration.

As shown in FIG. 5, the injection device 100 includes one accessory 260 including a storing cannula 261 to collect the second fraction of solution.

From the above disclosure it is clear that establishing the quantity of the solution to administer to the patient occurs:
ejecting one first fraction of the solution out of the tubular body 3;
isolating one second fraction of the solution in the tubular body 3;
isolating one third fraction of the solution inside the dosing reservoir 20.

In particular, the second fraction of solution is intended to be ejected from the tubular body 3 through the vent way 23 and be housed in the storing cannula 261 alongside the administration of the third fraction contained in the dosing reservoir 20.

The storing cannula 261 is provided with a first end 266 and a second end 267.

One passage is defined between the first 266 and the second 267 ends for the second fraction of solution.

One storing reservoir 261′ is defined between the first 266 and the second 267 ends for the second fraction of solution.

Preferably, the second end 267 of the storing cannula 261 is open to enable the emission of the air existing in the storing reservoir 261′, such air would otherwise prevent the second fraction of solution from entering. The cross-section of the storing cannula 261 is such to retain the second fraction of solution by capillarity inside the storing reservoir (261′) even though the second end 267 of the storing cannula 261 is opened.

As shown in FIGS. 4 and 10, the storing cannula 261 is engageable with the vent opening 23, and in particular the first end 266 is engageable with the vent channel 26 of the injection device 100.

Starting from the pre-administering configuration of FIG. 4, the vent opening 23 is opened by removing the plug 28. The storing cannula 261, and in particular the first end 266, is engaged with the vent opening 23, for example it is inserted in the vent channel 26 of the injection device 100.

The storing cannula 261 is long enough to house all the second fraction of solution.

Preferably, the second end 267 of the storing cannula 261 is housed into a housing 268 provided at the grasping portion 12.

As shown in FIGS. 10 and 11, the accessory 260 also includes one support 262 to guarantee to position the storing cannula 261 in the vent channel 26 of the injection device 100.

The support 262 is for example a plate provided with a first seat 263 for housing the storing cannula 261 and a second seat 264 for housing a vent channel 26.

The first 263 and the second 264 seats are for example holes, or grooves provided in the body 265 of the support 262.

As shown in FIG. 10, the vent channel 26 is housed in the second seat 264 provided in the support 262, and alongside in the vent channel 26 the first end 266 of the storing cannula 261 is housed. Such a construction choice improves the fixing of the storing cannula 261 to the injection device 100.

As shown in FIG. 6, the storing cannula 261 runs in parallel with the containment body 3 of the injection device 100 between the closing element 2 and the grasping portion 12. Such a construction choice makes the injection device 100 with storing cannula 261 particularly compact and convenient during the administration of the injectable solution S, as the storing cannula 261 does not hinder the physician's actions.

In order to run in parallel to the containment body 3, the storing cannula 261 is provided with a curved portion 269.

Preferably, the injection device 100 further includes a stopper 50 to the sliding of the plunger 52.

As shown in FIG. 12, the stopper 50 is applicable to the plunger 52, for example by interlocking and is preferably removable, and it is suitable to abut against the grasping portion 12.

The stopper 50 is suitable to abut against the grasping portion 12 at the configuration of FIG. 4, that is at the pre-administering configuration. Advantageously, the stopper 50 prevents the plunger 52 from further advancing during the assembling step of the storing cannula 261.

Advantageously, the stopper 50 prevents the plunger 52 from further advancing at a precise dose of solution S, for example at a dose equal to 0.1 ml of solution.

In the embodiment variant shown in the FIGS. 13 to 16, the injection device 100 is of the double-chamber type and includes:
one first containment chamber 8 suitable to contain a first solid or liquid substance P;
a first 6 and a second 7 plug, arranged inside the tubular body 3, so as to delimit the second containment chamber 9 therebetween the tubular body (3) and suitable to slide inside the tubular body 3 (for example as a result of a pushing or pulling force);
a second containment chamber 9 suitable to contain a second liquid substance L, intended to be mixed inside the tubular body 3 with the first substance P for the reconstitution of an injectable solution.

In the embodiment variant wherein the injection device 100 is of the pre-filled type, the first chamber 8 contains a first substance P and the second chamber 9 contains a second substance L.

Preferably, the first substance P is a highly active substance, for instance a powder, a substance in granules or a sterile tablet or compacted powder. In case the first substance P is solid, it may be a crystallized or freeze-dried substance.

Preferably, the aforesaid second liquid substance L is a solvent for injectable use.

According to one embodiment (not shown in the Figures), the first plug 6, also called intermediate plug 6, includes at least a bypass channel which is originally in a closing condition and which is suitable to be taken to an opening condition after an external force occurrence, e.g. a pressure force acting on the plug.

In the embodiment of FIG. 13, the containment body 3 includes one inner wall provided with a recess 10 suitable to define a bypass channel, arranged downstream from the first plug 6.

Starting from the initial configuration of FIG. 15, following a pushing action of the plunger 52, the first 6 and the second 7 plugs are suitable to slide in the tubular body 3 until they reach a so called reconstitution configuration, represented in FIG. 16, wherein two containment chambers 8 and 9 are in communication with each other through bypass 10 and the second substance 9 enters into the first chamber 8 to mix with the first substance.

Starting from the operative configuration of FIG. 16, by further pushing the plunger 52, it is possible to further advance both plugs 6 and 7 until said plugs contact each other, eject the air existing in the first containment chamber 8 until they reach the operative configuration of FIG. 3, also called “air-ejection end” configuration.

This invention also relates to an accessory 260 for an injection device 100, such accessory including at least one storing cannula 261 to collect one fraction of injectable solution S. Preferably, the accessory 260 also includes one support 262 suitable for guaranteeing the correct positioning of the storing cannula 261 on the injection device 100.

Innovatively, an injection device according to this invention avoids the dispersion of the ejected fraction during the injection step of the fraction of administration.

Advantageously, the storing cannula for containing the ejected fraction is easily applicable to the injection device with no need for substantial changes.

Advantageously, an injection device according to this invention enables the physician not to contact the pharmaceutical substance.

Advantageously, an injection device according to this invention allows to administer very precise and reduced doses of an injection solution, e.g. of about one millilitre or one tenth of millilitre.

Subject to the principle of the invention, its embodiments and implementation details shall widely vary with respect to what has been disclosed and illustrated for exemplary non-limiting purposes, without departing from the scope of protection as defined in the enclosed claims.

1. An injection device for an injectable solution, comprising:
a containment tubular body, closed at the front by a closing element and at the back by a grasping portion, inside which at least one containment chamber is defined;
a dosing reservoir suitable to contain a fraction of the injectable solution, and having an inlet opening in communication with the containment chamber and outlet opening;
at least one plug arranged inside the tubular body in order to delimit the containment chamber, said plug being suitable to slide inside the tubular body and including a projecting appendix suitable to pass through the inlet opening of the dosing reservoir and to enter inside the dosing reservoir in order to eject said fraction of solution from the dosing reservoir;
a vent outlet, provided with a vent channel, suitable to be passed through by a second fraction of solution to eject the second fraction and to allow the projecting appendix to pass through the inlet opening of the dosing reservoir; and
characterized by including an accessory having a storing cannula being engageable with the vent outlet, and provided with a first end and a second end between which a storing reservoir for the second fraction of solution is defined.

2. The injection device according to claim 1, wherein the cross-section of the storing cannula is configured to retain the second fraction of solution by capillarity inside the storing reservoir.

3. The injection device according to claim 1, wherein the storing cannula is long enough to house all the second fraction of solution.

4. The injection device according to claim 1, wherein the second end of the storing cannula is open in order to allow air existing in the storing reservoir to exit.

5. The injection device according to claim 1, wherein the first end of the storing cannula is inserted or insertable in the vent channel of the injection device.

6. The injection device according to claim 1, wherein the second end of the storing cannula is inserted or insertable in a housing being provided for at the grasping portion.

7. The injection device according to claim 1, wherein the storing cannula runs in parallel to the tubular body between the closing element and the grasping portion.

8. The injection device according to claim 1, wherein the accessory also comprises a support to position the storing cannula on the injection device, and wherein the support is provided with a first seat to house the storing cannula and a second seat to house the vent channel.

9. The injection device according to claim 1, comprising a plunger suitable to push the plug, and a stopper for sliding of the plunger, said stopper applicable by interlocking to the plunger and it is suitable to abut against the grasping portion in a pre-administering configuration.

10. The injection device according to claim 1, wherein said fraction is less than about 1/25 of the injectable solution.

11. A prefilled injection device with a single chamber, according to claim 1, wherein the containment chamber contains an injectable solution.

12. The injection device according to claim 1, comprising:
a first and a second containment chamber being hermetically separated from one another;
a first solid or liquid substance contained in the first containment chamber;
a first and a second plug, arranged inside the tubular body, so as to define the second containment chamber therebetween in the tubular body and adapted to slide inside the tubular body;
a second liquid substance, contained inside the second containment chamber, intended to be mixed inside the tubular body with the first substance to reconstitute an injectable solution; and
a bypass channel between the first and second containment chambers suitable to allow mixing the first substance and the second substance in order to reconstitute the injectable solution.
